(19) United States
(12) Patent Application Publication
(10) Pub. No.: US 2022/0101954 A1
(43) Pub. Date: Mar. 31, 2022

(54) METHOD FOR DRUG CLASSIFICATION, TERMINAL DEVICE, AND NON-TRANSITORY COMPUTER-READABLE STORAGE MEDIUM

(71) Applicant: Ping An Technology (Shenzhen) Co., Ltd., Shenzhen (CN)

(72) Inventors: Jun WANG, Shenzhen (CN); Pengyong LI, Shenzhen (CN)

(73) Assignee: Ping An Technology (Shenzhen) Co., Ltd., Shenzhen (CN)

(21) Appl. No.: 17/539,794

(22) Filed: Dec. 1, 2021

Related U.S. Application Data
(63) Continuation of application No. PCT/CN2020/124690, filed on Oct. 29, 2020.

(30) Foreign Application Priority Data
Sep. 27, 2020 (CN) 202011035837.1

(51) Int. Cl. G16C 20/20; G16H 70/40; G16H 50/20; G06N 3/04; G16C 20/70

(52) U.S. Cl. CPC G16C 20/20; G16H 70/40; G16C 20/70; G06N 3/04; G16H 50/20

(57) ABSTRACT

A method for drug classification, a terminal device, and a non-transitory computer-readable storage medium are provided. An attribute feature vector of each of n atoms in a drug molecule to be detected and an attribute feature vector of a virtual atom are obtained. An adjacency matrix is constructed according to a connection relationship between the virtual atom and each of the n atoms and between the n atoms. An atom attribute feature matrix is constructed according to the attribute feature vector of each atom. The adjacency matrix and the atom attribute feature matrix are inputted into a graph neural network to determine a transfer feature matrix of the n atoms and the virtual atom. A molecular feature vector corresponding to the drug molecule to be detected is determined according to the transfer feature matrix. The molecular feature vector is inputted into a classifier to output a drug category.

### CROSS-REFERENCE TO RELATED APPLICATION(S)

The application is a continuation under 35 U.S.C. § 120 of International Application No. PCT/CN2020/124690, filed on Oct. 29, 2020, which claims priority under 35 U.S.C. § 119(a) and/or PCT Article 8 to Chinese Patent Application No. 202011035837.1, filed on Sep. 27, 2020, the entire disclosures of which are hereby incorporated by reference.

### TECHNICAL FIELD

This disclosure relates to the field of data processing, and more particularly to a method for drug classification, a terminal device, and a non-transitory computer-readable storage medium.

### BACKGROUND

The drug development process has characteristics of high capital density, high risk, and long cycle, which requires a large amount of capital, and manpower and material resources. The inventor realized that although traditional machine learning methods can assist in drug development to a certain extent, the traditional machine learning methods require molecular descriptors as a feature input, and the selection of different molecular descriptors has a great impact on the performance of a machine learning model. Therefore, most traditional machine learning methods require complicated and tedious feature engineering. During research, the inventor found that emerging deep learning methods in recent years can directly extract features from original structures of drugs, so as to avoid feature engineering and shorten the development cycle. However, when features of drug molecules extracted based on the existing deep learning methods are used for predicting drug classification, the prediction accuracy still needs to be improved.

### SUMMARY

In a first aspect, implementations of the disclosure provide a method for drug classification. The method includes the following. An attribute feature vector of each of n atoms in a drug molecule to be detected and an attribute feature vector of a virtual atom are obtained. The virtual atom is connected with each of the n atoms. An adjacency matrix is constructed according to a connection relationship between the virtual atom and each of the n atoms and a connection relationship between the n atoms. An atom attribute feature matrix is constructed according to the attribute feature vector of each of the n atoms and the attribute feature vector of the virtual atom. The adjacency matrix and the atom attribute feature matrix are inputted into a graph neural network to determine a transfer feature matrix of the n atoms and the virtual atom through the graph neural network. A molecular feature vector corresponding to the drug molecule to be detected is determined according to the transfer feature matrix, and the molecular feature vector is inputted into a classifier to output a drug category of the drug molecule to be detected through the classifier.

In a second aspect, implementations of the disclosure provide a terminal device. The terminal device includes a processor and a memory coupled with the processor. The memory is configured to store computer programs. The computer programs include program instructions. The processor is configured to invoke the program instructions to perform the following. An attribute feature vector of each of n atoms in a drug molecule to be detected and an attribute feature vector of a virtual atom are obtained. The virtual atom is connected with each of the n atoms. An adjacency matrix is constructed according to a connection relationship between the virtual atom and each of the n atoms and a connection relationship between the n atoms, and an atom attribute feature matrix is constructed according to the attribute feature vector of each of the n atoms and the attribute feature vector of the virtual atom. The adjacency matrix and the atom attribute feature matrix are inputted into a graph neural network to determine a transfer feature matrix of the n atoms and the virtual atom through the graph neural network. A molecular feature vector corresponding to the drug molecule to be detected is determined according to the transfer feature matrix, and the molecular feature vector is inputted into a classifier to output a drug category of the drug molecule to be detected through the classifier.

In a third aspect, implementations of the disclosure provide a non-transitory computer-readable storage medium. The non-transitory computer-readable storage medium stores computer programs including program instructions. The program instructions which, when executed by a processor, cause the processor to perform the following. An attribute feature vector of each of n atoms in a drug molecule to be detected and an attribute feature vector of a virtual atom are obtained. The virtual atom is connected with each of the n atoms. An adjacency matrix is constructed according to a connection relationship between the virtual atom and each of the n atoms and a connection relationship between the n atoms, and an atom attribute feature matrix is constructed according to the attribute feature vector of each of the n atoms and the attribute feature vector of the virtual atom. The adjacency matrix and the atom attribute feature matrix are inputted into a graph neural network to determine a transfer feature matrix of the n atoms and the virtual atom through the graph neural network. A molecular feature vector corresponding to the drug molecule to be detected is determined according to the transfer feature matrix, and the molecular feature vector is inputted into a classifier to output a drug category of the drug molecule to be detected through the classifier.

### BRIEF DESCRIPTION OF THE DRAWINGS

To describe technical solutions of implementations of the disclosure more clearly, the following will give a brief description of accompanying drawings used for describing the implementations of the disclosure. Apparently, the accompanying drawings described in the following are merely some implementations of the disclosure. Those of ordinary skill in the art can also obtain other accompanying drawings based on the accompanying drawings described below without creative efforts.

FIG. 1 is a schematic flow chart illustrating a method for drug classification provided in implementations of the disclosure.

FIG. 2 is a schematic diagram illustrating a molecular structure of a drug molecule to be detected provided in implementations of the disclosure.

FIG. 3 is a schematic flow chart illustrating a method for drug classification provided in other implementations of the disclosure.

FIG. 4 is a schematic structural diagram illustrating an apparatus for drug classification provided in implementations of the disclosure.

FIG. 5 is a schematic structural diagram illustrating an apparatus for drug classification provided in other implementations of the disclosure.

FIG. 6 is a schematic structural diagram illustrating a terminal device provided in implementations of the disclosure.

### DETAILED DESCRIPTION

Technical solutions embodied in implementations of the disclosure will be described in a clear and comprehensive manner in conjunction with accompanying drawings of the implementations of the disclosure. It is evident that the implementations described herein are merely some of rather than all the implementations of the disclosure. All other implementations obtained by those of ordinary skill in the art based on the implementations of the disclosure without creative efforts shall fall within the protection scope of the disclosure.

The technical solutions of the disclosure can be applied to the technical fields of artificial intelligence (AI), digital medical, smart city, blockchain, and/or mega data, for example, the technical solutions specifically relate to the neural network technology, so as to improve the accuracy of drug classification, thereby realizing smart medical treatment. Optionally, in the disclosure involved data such as a feature vector(s) and/or a drug category(ies), can be stored in a database or a blockchain, and the disclosure is not limited thereto.

For example, the method for drug classification provided in implementations of the disclosure can be applied to the medical field. According to the method in implementations of the disclosure, by constructing an adjacency matrix and an atom attribute feature matrix of n atoms that constitute a drug molecule and a virtual atom, and inputting the constructed adjacency matrix and atom attribute feature matrix into a graph neural network for feature learning, a transfer feature matrix that corresponds to the n atoms and the virtual atom can be determined based on message transfer characteristics of the graph neural network. Thereafter, according to transfer feature vectors in the transfer feature matrix, a molecular feature vector corresponding to the drug molecule can be determined, and thus a drug category can be determined according to the molecule feature vector of the drug molecule. According to the implementations of the disclosure, the accuracy of the drug classification can be improved.

The following will respectively describe the method and related devices provided in implementations of the disclosure in detail with reference to FIGS. 1 to 6.

FIG. 1 is a schematic flow chart illustrating a method for drug classification provided in implementations of the disclosure. As illustrated in FIG. 1, the method provided in implementations of the disclosure includes operations at S101 to S104.

At S101, an attribute feature vector of each atom in a drug molecule to be detected and an attribute feature vector of a virtual atom are obtained.

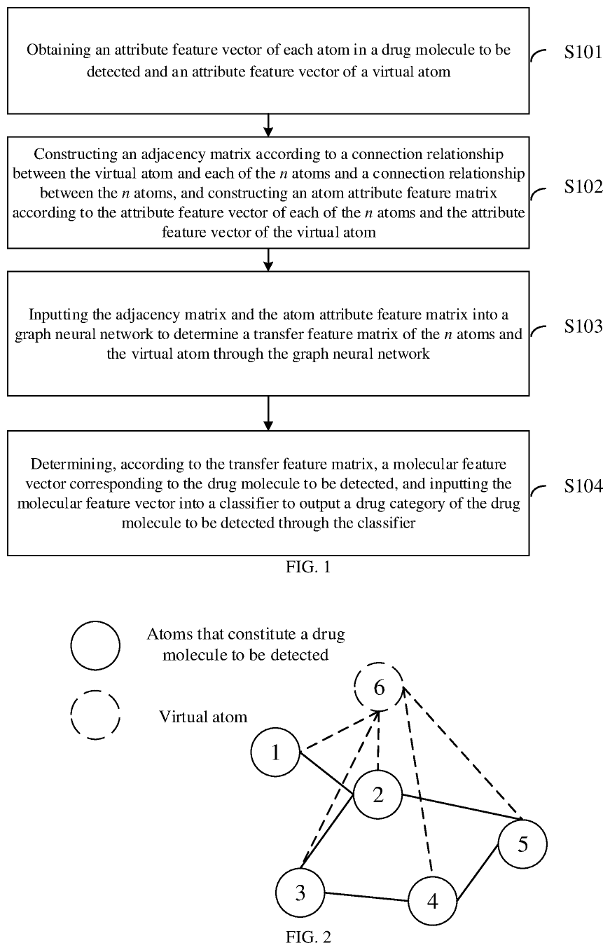

In some feasible implementations, to realize classification of a drug or multiple drugs, an attribute feature vector of each atom in a drug molecule of a drug to be classified (for convenience of description, “drug molecule to be detected” for short) and an attribute feature vector of a virtual atom can be obtained first. The drug molecule to be detected includes n atoms, and the virtual atom is connected with each of the n atoms, where n is an integer. It can be understood that the attribute feature vector of any atom among the n atoms and the virtual atom is determined according to an attribute feature of the any atom, and the attribute feature of the any atom includes but is not limited to one or more of an atom type, the number of chemical bonds, formal charge, atomic chirality, the number of connected hydrogen atoms, atomic orbital, and aromaticity. The virtual atom can be considered to be an atom outside the structure of the drug molecule to be detected. That is, the virtual atom is not an atom that actually constitutes the drug molecule to be detected, and the virtual atom is just an atom that has a connection relationship with each of the n atoms. For example, referring to FIG. 2, FIG. 2 is a schematic diagram illustrating a molecular structure of the drug molecule to be detected provided in implementations of the disclosure. As illustrated in FIG. 2, the drug molecule to be detected includes 5 atoms (i.e., n=5), which are atom 1, atom 2, atom 3, atom 4, and atom 5, respectively. Atom 1 is connected with atom 2. Atom 2 is also connected with atom 3 and atom 5, respectively. Atom 3 is connected with atom 4, and atom 4 is also connected with atom 5. The virtual atom is atom 6 as illustrated in FIG. 2. It can be seen from FIG. 2 that atom 6 is connected with each of the 5 atoms in the drug molecule to be detected.

At S102, an adjacency matrix is constructed according to a connection relationship between the virtual atom and each of the n atoms and a connection relationship between the n atoms, and an atom attribute feature matrix is constructed according to the attribute feature vector of each of the n atoms and the attribute feature vector of the virtual atom.

In some feasible implementations, the adjacency matrix can be constructed according to the connection relationship between the virtual atom and each of the n atoms and the connection relationship between the n atoms. In addition, the atom attribute feature matrix can be constructed according to the attribute feature vector of each of the n atoms and the attribute feature vector of the virtual atom. For example, as illustrated in FIG. 2, according to the connection relationship between the virtual atom and each of the 5 atoms and the connection relationship between the 5 atoms, adjacency matrix A can be constructed.

$$A = [ \begin{matrix}0 & 1 & 0 & 0 & 0 & 1 \\ 1 & 0 & 1 & 0 & 1 & 1 \\ 0 & 1 & 0 & 1 & 0 & 1 \\ 0 & 0 & 1 & 0 & 1 & 1 \\ 0 & 1 & 0 & 1 & 0 & 1 \\ 1 & 1 & 1 & 1 & 1 & 0\end{matrix} ]$$

Rows in adjacency matrix A respectively represent atom 1, atom 2, atom 3, atom 4, atom 5, and virtual atom 6 from top to bottom. Columns in adjacency matrix A also respectively represent atom 1, atom 2, atom 3, atom 4, atom 5, and virtual atom 6 from left to right. Assuming that the attribute feature of each atom includes F₁, F₂, F₃, . . . , Fₘ, atom attribute feature matrix F can be constructed according to the attribute feature vector corresponding to the attribute feature of each atom.

$$F = [ \begin{matrix}{x}_{11} & ⋯ & {x}_{1 m} \\ {x}_{21} & ⋯ & {x}_{2 m} \\ {x}_{31} & ⋯ & {x}_{3 m} \\ {x}_{41} & ⋯ & {x}_{4 m} \\ {x}_{51} & ⋯ & {x}_{5 m} \\ {x}_{61} & ⋯ & {x}_{6 m}\end{matrix} ]$$

Rows in atom attribute feature matrix F respectively represent atom 1, atom 2, atom 3, atom 4, atom 5, and virtual atom 6 from top to bottom. From left to right columns in atom attribute feature matrix F represent feature vectors x₁, x₂, x₃, . . . , xₘ respectively corresponding to attribute features F₁, F₂, F₃, . . . , Fₘ of atoms. That is, the columns in the atom attribute feature matrix represent the attribute features of the atoms, and each row in the atom attribute feature matrix corresponds to an attribute feature vector of an atom in the drug molecule to be detected. It can be understood that the attribute feature vector of any atom among the n atoms and the virtual atom is determined according to the attribute feature of the any atom. The attribute feature of the any atom includes one or more of an atom type, the number of chemical bonds, formal charge, atomic chirality, the number of connected hydrogen atoms, atomic orbital, and aromaticity, and the disclosure is not limited thereto.

At S103, the adjacency matrix and the atom attribute feature matrix are inputted into a graph neural network to determine a transfer feature matrix of the n atoms and the virtual atom through the graph neural network.

In some feasible implementations, by inputting the adjacency matrix and the atom attribute feature matrix that are constructed based on the above operations into the graph neural network, the transfer feature matrix of the n atoms and the virtual atom can be determined through the graph neural network. It is to be noted that the graph neural network is a connection model, which can obtain dependencies of the graph through message transfer between nodes in the graph. That is, the graph neural network model can update representation of a node by aggregating information from neighboring nodes of the node.

At S104, a molecular feature vector corresponding to the drug molecule to be detected is determined according to the transfer feature matrix, and the molecular feature vector is inputted into a classifier to output a drug category of the drug molecule to be detected through the classifier.

In some feasible implementations, after the transfer feature matrix of the n atoms and the virtual atom is determined, the molecular feature vector corresponding to the drug molecule to be detected is determined according to the transfer feature matrix. The transfer feature matrix includes (n+1) transfer feature vectors, and each atom corresponds to one transfer feature vector.

The molecular feature vector corresponding to the drug molecule to be detected may be a transfer feature vector corresponding to the virtual atom in the transfer feature matrix. In other words, the transfer feature vector corresponding to the virtual atom in the transfer feature matrix can be determined as the molecular feature vector corresponding to the drug molecule to be detected. Alternatively, a first molecular feature vector is determined according to n transfer feature vectors corresponding to the n atoms in the transfer feature matrix, and a second molecular feature vector is determined according to a transfer feature vector corresponding to the virtual atom in the transfer feature matrix. Thereafter, the molecular feature vector corresponding to the drug molecule to be detected is determined according to the first molecular feature vector and the second molecular feature vector. For example, a sum of the n transfer feature vectors corresponding to the n atoms in the transfer feature matrix is determined as the first molecular feature vector, and the transfer feature vector corresponding to the virtual atom in the transfer feature matrix is determined as the second molecular feature vector. By performing a weighted sum on the first molecular feature vector and the second molecular feature vector, a third molecular feature vector is obtained and then determined as the molecular feature vector corresponding to the drug molecule to be detected. It can be understood that for the weighted sum, a sum of a first weight parameter corresponding to the first molecular feature vector and a second weight parameter corresponding to the second molecular feature vector is equal to 1, and the first weight parameter is less than the second weight parameter. The values of the weight parameters can be determined according to actual needs, and the disclosure is not limited thereto.

Furthermore, by inputting the molecular feature vector into the classifier, the drug category of the drug molecule to be detected can be outputted through the classifier. It can be understood that in the disclosure, a training data set is obtained, and the graph neural network and the classifier are trained according to multiple drug molecule training samples in the training data set, so as to obtain the graph neural network and the classifier that satisfy a convergence condition. The training data set includes the multiple drug molecule training samples. Each drug molecule training sample includes at least one sample drug molecule and a drug category label for each of the at least one sample drug molecule. According to the at least one sample drug molecule, an adjacency matrix and an atom attribute feature matrix that correspond to the at least one sample drug molecule can be obtained. When the adjacency matrix and the atom attribute feature matrix are inputted into the graph neural network, a transfer feature matrix corresponding to the at least one sample drug molecule can be determined. Thereafter, according to the transfer feature matrix, a molecular feature vector corresponding to the at least one sample drug molecule is determined. When the molecular feature vector is inputted into the classifier, a predicted drug category of the at least one sample drug molecule can be outputted. If the predicted drug category is different from the drug category label in the drug molecule training sample, parameters of the graph neural network and the classifier are adjusted until the predicted drug category is consistent with the drug category label in the drug molecule training sample. It can be understood that the drug category label may include a first label and a second label. In other words, the drug classification in the disclosure may be a binary classification of the drug. The first label is used for indicating a type of a disease that can be treated by the medicine, and the second label is used for indicating a type of a disease that cannot be treated by the medicine. For example, to discover potential therapeutic drugs for novel corona virus disease 2019 (COVID-19), existing potential inhibitory drugs for COVID-19 can be used as a training data set, and then a trained model can be used to select other potential drug molecules for treating COVID-19 from a DrugBank database (including 10971 drugs). That is to say, by inputting the molecular feature vector into the classifier, the drug category of the drug molecule to be detected can be determined according to an output result of the classifier, and then whether the drug molecule can be used in clinical trials for treatment of COVID-19 can be determined. The classifier may include a feedforward neural network and the like, which is not limited herein.

In implementations of the disclosure, the attribute feature vector of each atom in the drug molecule to be detected and the attribute feature vector of the virtual atom are obtained. The adjacency matrix is constructed according to the connection relationship between the virtual atom and each of the n atoms and the connection relationship between the n atoms. The atom attribute feature matrix is constructed according to the attribute feature vector of each of the n atoms and the attribute feature vector of the virtual atom. The drug molecule to be detected includes the n atoms, and the virtual atom is connected with each of the n atoms. The adjacency matrix and the atom attribute feature matrix are inputted into the graph neural network to determine the transfer feature matrix of the n atoms and the virtual atom through the graph neural network. The molecular feature vector corresponding to the drug molecule to be detected is determined according to the transfer feature matrix. The molecular feature vector is inputted into the classifier to output the drug category of the drug molecule to be detected through the classifier. According to the implementations of the disclosure, the accuracy of the drug classification can be improved.

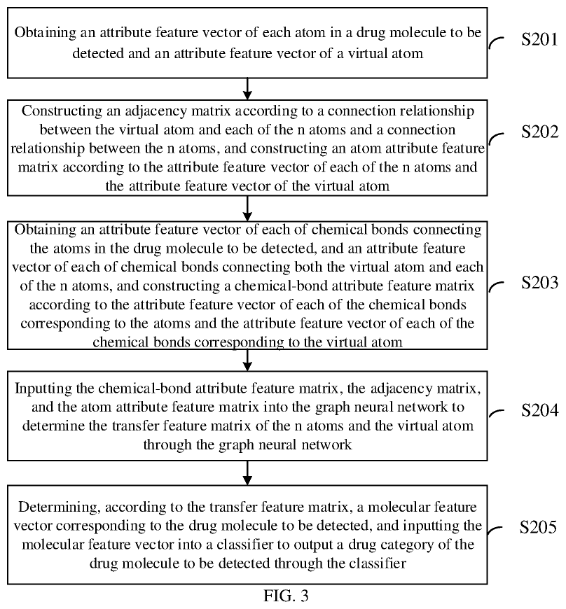

FIG. 3 is a schematic flow chart illustrating a method for drug classification provided in other implementations of the disclosure. As illustrated in FIG. 3, the method for drug classification provided in implementations of the disclosure can also be described according to implementations provided in the following S201 to S205.

At S201, an attribute feature vector of each atom in a drug molecule to be detected and an attribute feature vector of a virtual atom are obtained.

For operations at S201, reference may be made to the description of S101 in the corresponding implementations illustrated in FIG. 1, which will not be repeated herein.

At S202, an adjacency matrix is constructed according to a connection relationship between the virtual atom and each of the n atoms and a connection relationship between the n atoms, and an atom attribute feature matrix is constructed according to the attribute feature vector of each of the n atoms and the attribute feature vector of the virtual atom.

For operations at S202, reference may be made to the description of S102 in the corresponding implementations of FIG. 1, and will not be repeated herein.

At S203, an attribute feature vector of each of chemical bonds connecting the n atoms in the drug molecule to be detected and an attribute feature vector of each of chemical bonds connecting both the virtual atom and each of the n atoms are obtained, and a chemical-bond attribute feature matrix is constructed according to the attribute feature vector of each of the chemical bonds corresponding to the n atoms and the attribute feature vector of each of the chemical bonds corresponding to the virtual atom.

In some feasible implementations, by obtaining the attribute feature vector of each of the chemical bonds connecting the atoms in the drug molecule to be detected, and the attribute feature vector of each of the chemical bonds connecting both the virtual atom and each of the n atoms, the chemical-bond attribute feature matrix can be constructed according to the attribute feature vector of each of the chemical bonds corresponding to the atoms and the attribute feature vector of each of the chemical bonds corresponding to the virtual atom. The attribute feature vector of any chemical bond among the chemical bonds connecting the n atoms and the chemical bonds connecting both the virtual atom and each of the n atoms is determined according to an attribute feature of the any chemical bond. Assuming that the attribute feature of each chemical bond includes T1, T2, T3, . . . , Tb, chemical-bond attribute feature matrix T can be constructed according to the attribute feature vector corresponding to the attribute feature of each chemical bond.

$$T = [ \begin{matrix}{y}_{11} & ⋯ & {y}_{1 b} \\ {y}_{21} & ⋯ & {y}_{2 b} \\ {y}_{31} & ⋯ & {y}_{3 b} \\ {y}_{41} & ⋯ & {y}_{4 b} \\ ⋮ & ⋯ & ⋮ \\ {y}_{a 1} & ⋯ & {y}_{ab}\end{matrix} ]$$

Rows in the chemical-bond attribute feature matrix represent chemical bonds. Columns in the chemical-bond attribute feature matrix represent attribute features of the chemical bonds. That is, each row in the chemical-bond attribute feature matrix corresponds to an attribute feature vector of a chemical bond in the drug molecule to be detected. The attribute feature vector of any chemical bond is determined according to the attribute feature of the any chemical bond. It can be understood that the attribute feature of the any chemical bond includes one or more of a chemical bond type, a conjugate feature, a cyclic bond feature, and a molecular stereochemistry feature, and the disclosure is not limited thereto.

At S204, the chemical-bond attribute feature matrix, the adjacency matrix, and the atom attribute feature matrix are inputted into the graph neural network to determine the transfer feature matrix of the n atoms and the virtual atom through the graph neural network.

In some feasible implementations, by inputting the chemical-bond attribute feature matrix, the adjacency matrix, and the atom attribute feature matrix into the graph neural network, the transfer feature matrix of the n atoms and the virtual atom can be determined through the graph neural network. It can be understood that the graph neural network is a connection model, which can obtain dependencies of the graph through message transfer between nodes in the graph. That is, the graph neural network model can update representation of a node by aggregating information from neighboring nodes of the node. In addition, since the chemical bonds between atoms in the drug molecule to be detected can also carry different information, feature representation of each atom can be better learned according to the adjacency matrix, the atom attribute feature matrix, and the chemical-bond attribute feature matrix.

At S205, a molecular feature vector corresponding to the drug molecule to be detected is determined according to the transfer feature matrix, and the molecular feature vector is inputted into a classifier to output a drug category of the drug molecule to be detected through the classifier.

In some feasible implementations, after the transfer feature matrix of the n atoms and the virtual atom is determined, the molecular feature vector corresponding to the drug molecule to be detected is determined according to the transfer feature matrix. The transfer feature matrix includes (n+1) transfer feature vectors, and each atom corresponds to one transfer feature vector.

The molecular feature vector corresponding to the drug molecule to be detected may be a transfer feature vector corresponding to the virtual atom in the transfer feature matrix. In other words, the transfer feature vector corresponding to the virtual atom in the transfer feature matrix can be determined as the molecular feature vector corresponding to the drug molecule to be detected. Alternatively, a first molecular feature vector is determined according to n transfer feature vectors corresponding to the n atoms in the transfer feature matrix, and a second molecular feature vector is determined according to a transfer feature vector corresponding to the virtual atom in the transfer feature matrix. Thereafter, the molecular feature vector corresponding to the drug molecule to be detected is determined according to the first molecular feature vector and the second molecular feature vector. For example, a sum of the n transfer feature vectors corresponding to the n atoms in the transfer feature matrix is determined as the first molecular feature vector, and the transfer feature vector corresponding to the virtual atom in the transfer feature matrix is determined as the second molecular feature vector. By performing a weighted sum on the first molecular feature vector and the second molecular feature vector, a third molecular feature vector is obtained and then determined as the molecular feature vector corresponding to the drug molecule to be detected. It can be understood that during the weighted sum, a sum of a first weight parameter corresponding to the first molecular feature vector and a second weight parameter corresponding to the second molecular feature vector is equal to 1, and the first weight parameter is less than the second weight parameter. The value of the weight parameter can be determined according to actual needs, and the disclosure is not limited thereto.

Furthermore, by inputting the molecular feature vector into the classifier, the drug category of the drug molecule to be detected can be outputted through the classifier. It can be understood that in the disclosure, a training data set is obtained, and the graph neural network and the classifier are trained according to multiple drug molecule training samples included in the training data set, so as to obtain the graph neural network and the classifier that satisfy a convergence condition. The training data set includes the multiple drug molecule training samples. Each drug molecule training sample includes at least one sample drug molecule and a drug category label for each of the at least one sample drug molecule. It can be understood that the drug category label may include a first label and a second label. In other words, the drug classification in the disclosure can be a binary classification of the drug. The first label is used for indicating a type of a disease that can be treated by the medicine, and the second label is used for indicating a type of a disease that cannot be treated by the medicine. For example, to discover potential therapeutic drugs for novel corona virus disease, (COVID-19), existing potential inhibitory drugs for COVID-19 can be used as a training data set for learning of parameters of a model, and then a trained model can be used to screen (determine) other potential drug molecules for treating COVID-19 in a DrugBank database (including 10971 drugs). That is, by inputting the molecular feature vector into the classifier, the drug category of the drug molecule to be detected can be determined according to an output result of the classifier, and further determine whether the drug molecule can be used in clinical trials for treatment of COVID-19. The classifier may include a feedforward neural network and the like, which is not limited herein.

In implementations of the disclosure, the attribute feature vector of each atom in the drug molecule to be detected and the attribute feature vector of the virtual atom are obtained. The adjacency matrix is constructed according to the connection relationship between the virtual atom and each of the n atoms and the connection relationship between the n atoms. The atom attribute feature matrix is constructed according to the attribute feature vector of each of the n atoms and the attribute feature vector of the virtual atom. The drug molecule to be detected includes the n atoms, and the virtual atom is connected with each of the n atoms. The attribute feature vector of each of the chemical bonds connecting the atoms in the drug molecule to be detected and the attribute feature vector of each of the chemical bonds connecting the virtual atom and each of the n atoms are obtained. The chemical-bond attribute feature matrix is constructed according to the attribute feature vector of each of the chemical bonds corresponding to the atoms and the attribute feature vector of each of the chemical bonds corresponding to the virtual atom. The chemical-bond attribute feature matrix, the adjacency matrix, and the atom attribute feature matrix are inputted into the graph neural network to determine the transfer feature matrix of the n atoms and the virtual atom through the graph neural network. The molecular feature vector corresponding to the drug molecule to be detected is determined according to the transfer feature matrix. The molecular feature vector is inputted into the classifier to output the drug category of the drug molecule to be detected through the classifier. According to the implementations of the disclosure, the accuracy of the drug classification can be improved.

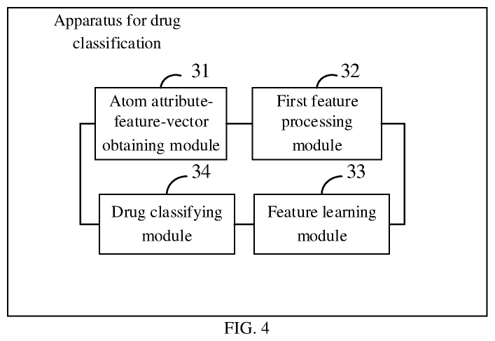

FIG. 4 is a schematic structural diagram illustrating an apparatus for drug classification provided in implementations of the disclosure. As illustrated in FIG. 4, the apparatus for drug classification includes an atom attribute-feature-vector obtaining module 31, a first feature processing module 32, a feature learning module 33, and a drug classifying module 34. The atom attribute-feature-vector obtaining module 31 is configured to obtain an attribute feature vector of each atom in a drug molecule to be detected and an attribute feature vector of a virtual atom, where the drug molecule to be detected includes n atoms, and the virtual atom is connected with each of the n atoms. The first feature processing module 32 is configured to construct an adjacency matrix according to a connection relationship between the virtual atom and each of the n atoms and a connection relationship between the n atoms, and construct an atom attribute feature matrix according to the attribute feature vector of each of the n atoms and the attribute feature vector of the virtual atom. The feature learning module 33 is configured to input the adjacency matrix and the atom attribute feature matrix into a graph neural network to determine a transfer feature matrix of the n atoms and the virtual atom through the graph neural network. The drug classifying module 34 is configured to determine, according to the transfer feature matrix, a molecular feature vector corresponding to the drug molecule to be detected, and input the molecular feature vector into a classifier to output a drug category of the drug molecule to be detected through the classifier.

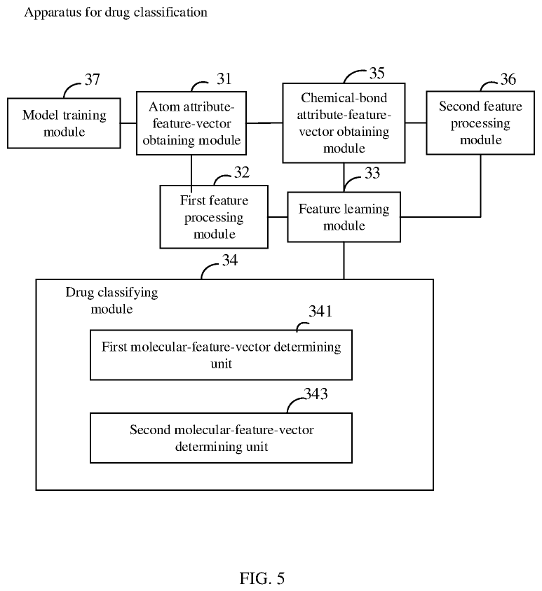

As illustrated in FIG. 5, FIG. 5 is a schematic structural diagram illustrating an apparatus for drug classification provided in other implementations of the disclosure.

In some feasible implementations, the attribute feature vector of any atom among the n atoms and the virtual atom is determined according to an attribute feature of the any atom. The attribute feature of the any atom includes one or more of an atom type, the number of chemical bonds, formal charge, atomic chirality, the number of connected hydrogen atoms, atomic orbital, and aromaticity.

In some feasible implementations, the apparatus further includes a chemical-bond attribute-feature-vector obtaining module 35 and a second feature processing module 36. The chemical-bond attribute-feature-vector obtaining module 35 is configured to obtain an attribute feature vector of each of chemical bonds connecting the n atoms in the drug molecule to be detected, and an attribute feature vector of each of chemical bonds connecting both the virtual atom and each of the n atoms. The second feature processing module 36 is configured to construct a chemical-bond attribute feature matrix according to the attribute feature vector of each of the chemical bonds corresponding to the n atoms and the attribute feature vector of each of the chemical bonds corresponding to the virtual atom. The feature learning module 33 is configured to input the chemical-bond attribute feature matrix, the adjacency matrix, and the atom attribute feature matrix into the graph neural network to determine the transfer feature matrix of the n atoms and the virtual atom through the graph neural network.

In some feasible implementations, the attribute feature vector of any chemical bond among the chemical bonds connecting the n atoms and the chemical bonds connecting the virtual atom and each of the n atoms is determined according to an attribute feature of the any chemical bond. The attribute feature of the any chemical bond includes one or more of a chemical bond type, a conjugate feature, a cyclic bond feature, and a molecular stereochemistry feature.

In some feasible implementations, the apparatus further includes a model training module 37. The model training module 37 is configured to obtain a training data set, where the training data set includes multiple drug molecule training samples. Each drug molecule training sample includes at least one sample drug molecule and a drug category label for each of the at least one sample drug molecule. The model training module 37 is further configured to train the graph neural network and the classifier according to the multiple drug molecule training samples to obtain the graph neural network and the classifier that satisfy a convergence condition.

In some feasible implementations, the drug classifying module 34 includes a first molecular-feature-vector determining unit 341. The first molecular-feature-vector determining unit 341 is configured to determine a transfer feature vector corresponding to the virtual atom in the transfer feature matrix as the molecular feature vector corresponding to the drug molecule to be detected.

In some feasible implementations, the drug classifying module 34 further includes a second molecular-feature-vector determining unit 343. The second molecular-feature-vector determining unit 343 is configured to obtain n transfer feature vectors corresponding to the n atoms in the transfer feature matrix, where each of the n atoms corresponds to one transfer feature vector. The second molecular-feature-vector determining unit 343 is further configured to determine a sum of the n transfer feature vectors as a first molecular feature vector, and determine a transfer feature vector corresponding to the virtual atom in the transfer feature matrix as a second molecular feature vector. The second molecular-feature-vector determining unit 343 is further configured to perform a weighted sum on the first molecular feature vector and the second molecular feature vector to obtain a third molecular feature vector, and determine the third molecular feature vector as the molecular feature vector corresponding to the drug molecule to be detected.

According to the apparatus in implementations of the disclosure, the attribute feature vector of each atom in the drug molecule to be detected and the attribute feature vector of the virtual atom are obtained. The adjacency matrix is constructed according to the connection relationship between the virtual atom and each of the n atoms and the connection relationship between the n atoms. The atom attribute feature matrix is constructed according to the attribute feature vector of each of the n atoms and the attribute feature vector of the virtual atom. The drug molecule to be detected includes the n atoms, and the virtual atom is connected with each of the n atoms. The attribute feature vector of each of chemical bonds connecting the atoms in the drug molecule to be detected and the attribute feature vector of each of chemical bonds connecting both the virtual atom and each of the n atoms are obtained. The chemical-bond attribute feature matrix is constructed according to the attribute feature vector of each of the chemical bonds corresponding to the atoms and the attribute feature vector of each of the chemical bonds corresponding to the virtual atom. The chemical-bond attribute feature matrix, the adjacency matrix, and the atom attribute feature matrix are inputted into the graph neural network to determine the transfer feature matrix of the n atoms and the virtual atom through the graph neural network. The molecular feature vector corresponding to the drug molecule to be detected is determined according to the transfer feature matrix. The molecular feature vector is inputted into the classifier to output the drug category of the drug molecule to be detected through the classifier. According to the implementations of the disclosure, the accuracy of the drug classification can be improved.

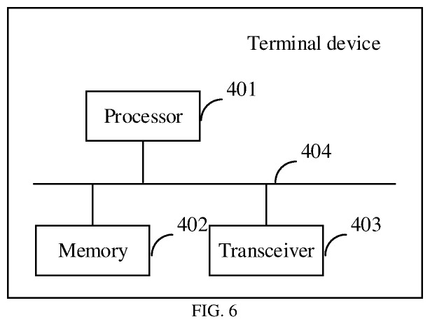

FIG. 6 is a schematic structural diagram illustrating a terminal device provided in implementations of the disclosure. As illustrated in FIG. 6, the terminal device may include at least one processor 401 and memory 402. The terminal device further includes a transceiver 403. The processor 401 and the memory 402 are coupled to each other, for example, the processor 401 and the memory 402 are coupled to each other via a bus 404. The memory 402 is configured to store computer programs. The computer programs include program instructions. The processor 401 is configured to invoke the program instructions to perform the following operations. An attribute feature vector of each atom in a drug molecule to be detected and an attribute feature vector of a virtual atom are obtained, where the drug molecule to be detected includes n atoms, and the virtual atom is connected with each of the n atoms. An adjacency matrix is constructed according to a connection relationship between the virtual atom and each of the n atoms and a connection relationship between the n atoms. An atom attribute feature matrix is constructed according to the attribute feature vector of each of the n atoms and the attribute feature vector of the virtual atom. The adjacency matrix and the atom attribute feature matrix are inputted into a graph neural network to determine a transfer feature matrix of the n atoms and the virtual atom through the graph neural network. A molecular feature vector corresponding to the drug molecule to be detected is determined according to the transfer feature matrix. The molecular feature vector is inputted into a classifier to output a drug category of the drug molecule to be detected through the classifier.

In some feasible implementations, the attribute feature vector of any atom among the n atoms and the virtual atom is determined according to an attribute feature of the any atom. The attribute feature of the any atom includes one or more of an atom type, the number of chemical bonds, formal charge, atomic chirality, the number of connected hydrogen atoms, atomic orbital, and aromaticity.

In some feasible implementations, the processor 401 is further configured to obtain an attribute feature vector of each of chemical bonds connecting the n atoms in the drug molecule to be detected, and an attribute feature vector of each of chemical bonds connecting both the virtual atom and each of the n atoms. The processor 401 is further configured to construct a chemical-bond attribute feature matrix according to the attribute feature vector of each of the chemical bonds corresponding to the n atoms and the attribute feature vector of each of the chemical bonds corresponding to the virtual atom.

The processor 401 is further configured to input the chemical-bond attribute feature matrix, the adjacency matrix, and the atom attribute feature matrix into the graph neural network to determine the transfer feature matrix of the n atoms and the virtual atom through the graph neural network.

In some feasible implementations, the attribute feature vector of any chemical bond among the chemical bonds connecting the n atoms and the chemical bonds connecting the virtual atom and each of the n atoms is determined according to an attribute feature of the any chemical bond. The attribute feature of the any chemical bond includes one or more of a chemical bond type, a conjugate feature, a cyclic bond feature, and a molecular stereochemistry feature.

In some feasible implementations, the processor 401 is further configured to obtain a training data set, where the training data set includes multiple drug molecule training samples. Each drug molecule training sample includes at least one sample drug molecule and a drug category label for each of the at least one sample drug molecule. The processor 401 is further configured to train the graph neural network and the classifier according to the multiple drug molecule training samples to obtain the graph neural network and the classifier that satisfy a convergence condition.

In some feasible implementations, the processor 401 is configured to determine a transfer feature vector corresponding to the virtual atom in the transfer feature matrix as the molecular feature vector corresponding to the drug molecule to be detected.

In some feasible implementations, the processor 401 is configured to obtain n transfer feature vectors corresponding to the n atoms in the transfer feature matrix, where each of the n atoms corresponds to one transfer feature vector. The processor 401 is further configured to determine a sum of the n transfer feature vectors as a first molecular feature vector, and determine a transfer feature vector corresponding to the virtual atom in the transfer feature matrix as a second molecular feature vector. The processor 401 is further configured to perform a weighted sum on the first molecular feature vector and the second molecular feature vector to obtain a third molecular feature vector, and determine the third molecular feature vector as the molecular feature vector corresponding to the drug molecule to be detected.

In some implementations, the processor 401 may be a central processing unit (CPU). The processor may also be other general-purpose processors, digital signal processors (DSP), application specific integrated circuits (ASIC), field-programmable gate arrays (FPGA), or other programmable logic devices, discrete gates, or transistor logic devices, discrete hardware components, or the like. The general-purpose processor may be a microprocessor or any conventional processor, or the like. The at least one memory 402 may include a read-only memory and a random access memory, and be configured to provide instructions and data to the processor 401. The at least one memory 402 may further include a non-transitory random access memory. For example, the memory 402 may store device-type information.

In implementations, the above-mentioned terminal device can execute the implementations provided in the steps in FIGS. 1 to 3 through built-in functional modules of the terminal device. For specific details, reference may be made to the implementations provided in the above-mentioned steps, which will not be repeated herein.

According to the terminal device of implementations of the disclosure, the attribute feature vector of each atom in the drug molecule to be detected and the attribute feature vector of the virtual atom are obtained. The adjacency matrix is constructed according to the connection relationship between the virtual atom and each of the n atoms and the connection relationship between the n atoms. The atom attribute feature matrix is constructed according to the attribute feature vector of each of the n atoms and the attribute feature vector of the virtual atom. The drug molecule to be detected includes the n atoms, and the virtual atom is connected with each of the n atoms. The attribute feature vector of each of chemical bonds connecting the atoms in the drug molecule to be detected and the attribute feature vector of each of chemical bonds connecting both the virtual atom and each of the n atoms are obtained. The chemical-bond attribute feature matrix is constructed according to the attribute feature vector of each of the chemical bonds corresponding to the atoms and the attribute feature vector of each of the chemical bonds corresponding to the virtual atom. The chemical-bond attribute feature matrix, the adjacency matrix, and the atom attribute feature matrix are inputted into the graph neural network to determine the transfer feature matrix of the n atoms and the virtual atom through the graph neural network. The molecular feature vector corresponding to the drug molecule to be detected is determined according to the transfer feature matrix. The molecular feature vector is inputted into the classifier to output the drug category of the drug molecule to be detected through the classifier. According to the implementations of the disclosure, the accuracy of the drug classification can be improved.

Implementations of the disclosure provide a computer-readable storage medium. The computer-readable storage medium stores computer programs, and the computer programs include program instructions which, when executed by a processor, cause the processor to implement the method provided in each step in FIG. 1 to FIG. 3. For specific details, reference may be made to implementations provided in the above operations, which will not be repeated herein.

In one example, the medium provided in implementations of the disclosure, such as the computer-readable storage medium, is a non-transitory computer-readable storage medium or a transitory computer-readable storage medium.

The computer-readable storage medium may be internal storage unit of the apparatus or the terminal device provided in any of the foregoing implementations, such as the hard disk or memory of an electronic device. The computer-readable storage medium may also be an external storage device of the electronic device, such as a plug-in hard disk, a smart media card (SMC), a secure digital (SD) card, or a flash card equipped on the electronic device. In addition, the computer-readable storage medium may also include both the internal storage unit of the electronic device and the external storage device. The computer-readable storage medium is configured to store the computer programs and other programs and data required by the electronic device. The computer-readable storage medium can also be configured to temporarily store data that has been output or will be outputted.

The terms “first”, “second”, “third”, “fourth”, and the like used in the specification, the claims, and the accompany drawings of the disclosure are used to distinguish different objects rather than describe a particular order. The terms “include”, “comprise”, and “have” as well as variations thereof are intended to cover non-exclusive inclusion. For example, a process, method, system, product, or apparatus including a series of steps or units is not limited to the listed steps or units, on the contrary, it can optionally include other steps or units that are not listed; alternatively, other steps or units inherent to the process, method, product, or device can be included either.

The term “implementation” referred to herein means that a particular feature, structure, or feature described in conjunction with the implementation may be contained in at least one implementation of the disclosure. The phrase appearing in various places in the specification does not necessarily refer to the same implementations, nor does it refer to an independent or alternative implementation that is mutually exclusive with other implementations. It is expressly and implicitly understood by those skilled in the art that an implementation described herein may be combined with other implementations. The term “and/or” used in the specification of the disclosure and the appended claims refers to any combination and all possible combinations of one or more of the associated listed items, and includes these combinations. Those of ordinary skill in the art will appreciate that units and algorithmic operations of various examples described in connection with implementations herein can be implemented by electronic hardware, by computer software, or by a combination of computer software and electronic hardware. In order to clearly explain interchangeability of hardware and software, in the above description, configurations and operations of each example have been generally described according to functions. Whether these functions are performed by means of hardware or software depends on the application and the design constraints of the associated technical solution. Those skilled in the art may use different methods for each particular application to implement the described functionality, but such methods should not be regarded as lying beyond the scope of the disclosure.

The methods and related devices provided in the implementations of the disclosure are described with reference to the method flowcharts and/or structural schematic diagrams provided in the implementations of the disclosure. Specifically, each process and/or or a block in the method flowcharts and/or structural schematics, or a combination of processes and/or blocks in the flowcharts and/or block diagrams can be implemented by computer program instructions. These computer program instructions can be provided to a processor of a general-purpose computer, a special-purpose computer, an embedded processor, or other programmable data processing equipment to generate a machine, so that the instructions executed by the processor of the computer or other programmable data processing equipment can produce an apparatus that realizes the functions specified in one block or multiple blocks in a flow chart or multiple flows and/or a schematic structural diagram. These computer program instructions can also be stored in a computer-readable memory that can guide a computer or other programmable data processing equipment to work in a specific manner, so that the instructions stored in the computer-readable memory produce an article of manufacture including the instruction device. The instruction apparatus realizes the functions specified in one process or multiple processes in the flowchart and/or one block or multiple blocks in the schematic structural diagram. These computer program instructions can also be loaded on a computer or other programmable data processing equipment, so that a series of operation steps are executed on the computer or other programmable equipment to produce computer-implemented processing, and the instructions executed execute on the computer or other programmable equipment can provide steps for implementing the functions specified in one block or multiple blocks in the flow chart or the flow chart and/or the structure.

What is claimed is:

1. A method for drug classification, comprising:
obtaining an attribute feature vector of each of n atoms in a drug molecule to be detected and an attribute feature vector of a virtual atom, wherein the virtual atom is connected with each of then atoms;
constructing an adjacency matrix according to a connection relationship between the virtual atom and each of the n atoms and a connection relationship between the n atoms, and constructing an atom attribute feature matrix according to the attribute feature vector of each of the n atoms and the attribute feature vector of the virtual atom;
inputting the adjacency matrix and the atom attribute feature matrix into a graph neural network to determine a transfer feature matrix of the n atoms and the virtual atom through the graph neural network; and
determining, according to the transfer feature matrix, a molecular feature vector corresponding to the drug molecule to be detected, and inputting the molecular feature vector into a classifier to output a drug category of the drug molecule to be detected through the classifier.

2. The method of claim 1, wherein the attribute feature vector of any atom among the n atoms and the virtual atom is determined according to an attribute feature of the any atom, wherein the attribute feature of the any atom comprises one or more of an atom type, the number of chemical bonds, formal charge, atomic chirality, the number of connected hydrogen atoms, atomic orbital, and aromaticity.

3. The method of claim 1, further comprising:
obtaining an attribute feature vector of each of chemical bonds connecting the n atoms in the drug molecule to be detected, and an attribute feature vector of each of chemical bonds connecting both the virtual atom and each of the n atoms; and
constructing a chemical-bond attribute feature matrix according to the attribute feature vector of each of the chemical bonds corresponding to the n atoms and the attribute feature vector of each of the chemical bonds corresponding to the virtual atom, wherein
inputting the adjacency matrix and the atom attribute feature matrix into the graph neural network to determine the transfer feature matrix of the n atoms and the virtual atom through the graph neural network comprises:
inputting the chemical-bond attribute feature matrix, the adjacency matrix, and the atom attribute feature matrix into the graph neural network to determine the transfer feature matrix of the n atoms and the virtual atom through the graph neural network.

4. The method of claim 3, wherein the attribute feature vector of any chemical bond among the chemical bonds connecting the n atoms and the chemical bonds connecting the virtual atom and each of then atoms is determined according to an attribute feature of the any chemical bond, wherein the attribute feature of the any chemical bond comprises one or more of a chemical bond type, a conjugate feature, a cyclic bond feature, and a molecular stereochemistry feature.

5. The method of claim 1, further comprising:
prior to obtaining the attribute feature vector of each atom in the drug molecule to be detected and the attribute feature vector of the virtual atom,
obtaining a training data set, wherein the training data set comprises a plurality of drug molecule training samples, wherein each drug molecule training sample comprises at least one sample drug molecule and a drug category label for each of the at least one sample drug molecule; and
training the graph neural network and the classifier according to the plurality of drug molecule training samples to obtain the graph neural network and the classifier that satisfy a convergence condition.

6. The method of claim 1, wherein determining, according to the transfer feature matrix, the molecular feature vector corresponding to the drug molecule to be detected comprises:
determining a transfer feature vector corresponding to the virtual atom in the transfer feature matrix as the molecular feature vector corresponding to the drug molecule to be detected.

7. The method of claim 1, wherein determining, according to the transfer feature matrix, the molecular feature vector corresponding to the drug molecule to be detected comprises:
obtaining n transfer feature vectors corresponding to the n atoms in the transfer feature matrix, wherein each of the n atoms corresponds to one transfer feature vector;
determining a sum of the n transfer feature vectors as a first molecular feature vector, and determining a transfer feature vector corresponding to the virtual atom in the transfer feature matrix as a second molecular feature vector; and
performing a weighted sum on the first molecular feature vector and the second molecular feature vector to obtain a third molecular feature vector, and determining the third molecular feature vector as the molecular feature vector corresponding to the drug molecule to be detected.

8. A terminal device, comprising:
a processor; and
a memory coupled with the processor and configured to store computer programs, wherein the computer programs comprise program instructions, and the processor is configured to invoke the program instructions to:
obtain an attribute feature vector of each of n atoms in a drug molecule to be detected and an attribute feature vector of a virtual atom, wherein the virtual atom is connected with each of the n atoms;
construct an adjacency matrix according to a connection relationship between the virtual atom and each of the n atoms and a connection relationship between the n atoms, and construct an atom attribute feature matrix according to the attribute feature vector of each of the n atoms and the attribute feature vector of the virtual atom;
input the adjacency matrix and the atom attribute feature matrix into a graph neural network to determine a transfer feature matrix of the n atoms and the virtual atom through the graph neural network; and
determine, according to the transfer feature matrix, a molecular feature vector corresponding to the drug molecule to be detected, and input the molecular feature vector into a classifier to output a drug category of the drug molecule to be detected through the classifier.

9. The terminal device of claim 8, wherein the attribute feature vector of any atom among the n atoms and the virtual atom is determined according to an attribute feature of the any atom, wherein the attribute feature of the any atom comprises one or more of an atom type, the number of chemical bonds, formal charge, atomic chirality, the number of connected hydrogen atoms, atomic orbital, and aromaticity.

10. The terminal device of claim 8, wherein the processor is further configured to:
obtain an attribute feature vector of each of chemical bonds connecting the n atoms in the drug molecule to be detected, and an attribute feature vector of each of chemical bonds connecting both the virtual atom and each of the n atoms; and
construct a chemical-bond attribute feature matrix according to the attribute feature vector of each of the chemical bonds corresponding to the n atoms and the attribute feature vector of each of the chemical bonds corresponding to the virtual atom, wherein
the processor configured to input the adjacency matrix and the atom attribute feature matrix into the graph neural network to determine the transfer feature matrix of the n atoms and the virtual atom through the graph neural network is configured to:
input the chemical-bond attribute feature matrix, the adjacency matrix, and the atom attribute feature matrix into the graph neural network to determine the transfer feature matrix of the n atoms and the virtual atom through the graph neural network.

11. The terminal device of claim 10, wherein the attribute feature vector of any chemical bond among the chemical bonds connecting the n atoms and the chemical bonds connecting the virtual atom and each of the n atoms is determined according to an attribute feature of the any chemical bond, wherein the attribute feature of the any chemical bond comprises one or more of a chemical bond type, a conjugate feature, a cyclic bond feature, and a molecular stereochemistry feature.

12. The terminal device of claim 8, wherein the processor is further configured to:
prior to obtaining the attribute feature vector of each atom in the drug molecule to be detected and the attribute feature vector of the virtual atom,
obtain a training data set, wherein the training data set comprises a plurality of drug molecule training samples, wherein each drug molecule training sample comprises at least one sample drug molecule and a drug category label for each of the at least one sample drug molecule; and
train the graph neural network and the classifier according to the plurality of drug molecule training samples to obtain the graph neural network and the classifier that satisfy a convergence condition.

13. The terminal device of claim 8, wherein the processor configured to determine, according to the transfer feature matrix, the molecular feature vector corresponding to the drug molecule to be detected is configured to:
determine a transfer feature vector corresponding to the virtual atom in the transfer feature matrix as the molecular feature vector corresponding to the drug molecule to be detected.

14. The terminal device of claim 8, wherein the processor configured to determine, according to the transfer feature matrix, the molecular feature vector corresponding to the drug molecule to be detected is configured to:
obtain n transfer feature vectors corresponding to the n atoms in the transfer feature matrix, wherein each of the n atoms corresponds to one transfer feature vector;
determine a sum of the n transfer feature vectors as a first molecular feature vector, and determine a transfer feature vector corresponding to the virtual atom in the transfer feature matrix as a second molecular feature vector; and
perform a weighted sum on the first molecular feature vector and the second molecular feature vector to obtain a third molecular feature vector, and determine the third molecular feature vector as the molecular feature vector corresponding to the drug molecule to be detected.

15. A non-transitory computer-readable storage medium storing computer programs, wherein the computer programs comprise program instructions which, when executed by a processor, cause the processor to:
obtain an attribute feature vector of each of n atoms in a drug molecule to be detected and an attribute feature vector of a virtual atom, wherein the virtual atom is connected with each of the n atoms;
construct an adjacency matrix according to a connection relationship between the virtual atom and each of the n atoms and a connection relationship between the n atoms, and construct an atom attribute feature matrix according to the attribute feature vector of each of the n atoms and the attribute feature vector of the virtual atom;
input the adjacency matrix and the atom attribute feature matrix into a graph neural network to determine a transfer feature matrix of the n atoms and the virtual atom through the graph neural network; and
determine, according to the transfer feature matrix, a molecular feature vector corresponding to the drug molecule to be detected, and input the molecular feature vector into a classifier to output a drug category of the drug molecule to be detected through the classifier.

16. The non-transitory computer-readable storage medium of claim 15, wherein the attribute feature vector of any atom among the n atoms and the virtual atom is determined according to an attribute feature of the any atom, wherein the attribute feature of the any atom comprises one or more of an atom type, the number of chemical bonds, formal charge, atomic chirality, the number of connected hydrogen atoms, atomic orbital, and aromaticity.

17. The non-transitory computer-readable storage medium of claim 15, wherein the program instructions, when executed by the processor, further cause the processor to:
obtain an attribute feature vector of each of chemical bonds connecting the n atoms in the drug molecule to be detected, and an attribute feature vector of each of chemical bonds connecting both the virtual atom and each of the n atoms; and
construct a chemical-bond attribute feature matrix according to the attribute feature vector of each of the chemical bonds corresponding to the n atoms and the attribute feature vector of each of the chemical bonds corresponding to the virtual atom, wherein
the program instructions executed by the processor to input the adjacency matrix and the atom attribute feature matrix into the graph neural network to determine the transfer feature matrix of the n atoms and the virtual atom through the graph neural network are executed by the processor to:
input the chemical-bond attribute feature matrix, the adjacency matrix, and the atom attribute feature matrix into the graph neural network to determine the transfer feature matrix of the n atoms and the virtual atom through the graph neural network.

18. The non-transitory computer-readable storage medium of claim 15, wherein the program instructions, when executed by the processor, further cause the processor to:
prior to obtaining the attribute feature vector of each atom in the drug molecule to be detected and the attribute feature vector of the virtual atom,
obtain a training data set, wherein the training data set comprises a plurality of drug molecule training samples, wherein each drug molecule training sample comprises at least one sample drug molecule and a drug category label for each of the at least one sample drug molecule; and
train the graph neural network and the classifier according to the plurality of drug molecule training samples to obtain the graph neural network and the classifier that satisfy a convergence condition.

19. The non-transitory computer-readable storage medium of claim 15, wherein the program instructions executed by the processor to determine, according to the transfer feature matrix, the molecular feature vector corresponding to the drug molecule to be detected are executed by the processor to:
determine a transfer feature vector corresponding to the virtual atom in the transfer feature matrix as the molecular feature vector corresponding to the drug molecule to be detected.

20. The non-transitory computer-readable storage medium of claim 15, wherein the program instructions executed by the processor to determine, according to the transfer feature matrix, the molecular feature vector corresponding to the drug molecule to be detected are executed by the processor to:
obtain n transfer feature vectors corresponding to the n atoms in the transfer feature matrix, wherein each of the n atoms corresponds to one transfer feature vector;
determine a sum of the n transfer feature vectors as a first molecular feature vector, and determine a transfer feature vector corresponding to the virtual atom in the transfer feature matrix as a second molecular feature vector; and
perform a weighted sum on the first molecular feature vector and the second molecular feature vector to obtain a third molecular feature vector, and determine the third molecular feature vector as the molecular feature vector corresponding to the drug molecule to be detected.
